(19) United States
(12) Patent Application Publication
(10) Pub. No.: US 2006/0063162 A1
(43) Pub. Date: Mar. 23, 2006

(54) Biological marker for inflammation

(76) Inventor: David X. Deng, Mountain View, GA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/948,716

(22) Filed: Sep. 23, 2004

(51) Int. Cl. C12Q 1/68; G01N 33/53; C12M 1/34

(52) U.S. Cl. 435/6; 435/7.1; 435/287.2

(57) ABSTRACT

The present disclosure provides methods and compositions for the diagnosis and treatment of inflammation, in particular, vascular pathologies. One aspect provides an array capable of detecting the expression pregnancy specific glycoproteins in a non-pregnant patient. The array optionally detects at least a second biomarker for vascular pathology. Compositions and methods including modulators of pregnancy specific glycoproteins are also provided.

### BACKGROUND

1. Technical Field

This disclosure relates generally to uses of pregnancy-specific glycoprotein as a marker and therapeutic target for inflammatory conditions, and in particular, for atherosclerosis.

2. Relevant Art

Vascular pathologies, including atherosclerosis, coronary heart disease, and stroke, are responsible for more than half of the yearly mortality in the United States, and more than 500,000 people die annually of myocardial infarction alone. This rate of mortality costs the United States more than $100 billion a year. More than 50 million people in the United States are candidates for some form of dietary and/or drug treatment to modify their lipid profile.

Monitoring lipid profiles in patients remains the primary method for diagnosing and monitoring atherosclerosis progression. Such methods focus on lipid levels such as cholesterol levels rather than on the vascular tissue itself. In particular, elevated low density lipoprotein (LDL) levels are generally accepted as an indicator of atherosclerosis.

Other biological indicators of atherosclerosis are known in the art. For example, C-reactive protein (CRP) levels are also used as a predictor of peripheral vascular disease. Serum amyloid A and soluble adhesion factors are other proteins that have been proposed as biomarkers for vascular inflammation. Recently, another class of secreted proteins called Pregnancy-associated plasma protein A (PAPP-A) has been suggested to be a biomarker for vascular inflammation.

PAPP-A is a large zinc-binding metalloproteinase of placental origin but physiologically present in men and women. The maternal serum level of PAPP-A increases exponentially until term. PAPP-A is found in the ovarian follicles, follicular fluid, luteal cells, and fallopian tubes of non-pregnant women and in the seminal vesicles and seminal fluid of males. Because low serum levels of PAPP-A have been demonstrated in first-trimester pregnancies associated with chromosomally abnormal fetuses, PAPP-A, with activity of a pro-atherosclerotic metalloproteinase, has been suggested as a potential biochemical marker for such pregnancies. Recent evidence has suggested that PAPP-A is involved in the development of atherosclerosis and is a new biomarker for unstable angina and acute myocardial infarction (7-10).

Despite the existence of multiple biomarkers suggestive of vascular disease, there remains a need for new and effective methods for diagnosing, detecting, treating, or preventing vascular disease.

### SUMMARY

It has been discovered that Pregnancy Specific Glycoproteins (PSG), a family of highly similar secreted proteins initially isolated from human placenta, are expressed in vascular smooth muscle cells and endothelial cells. Additionally, PSGs have been found to respond to atherogenic stimuli. Accordingly, one aspect of the present disclosure provides methods and compositions for the diagnosis and treatment of vascular pathology.

Another aspect provides an array having a first binding agent bound to a surface of the array. The first binding agent can be a nucleic acid complementary to a mRNA encoding PSG, or the first binding agent can be a polypeptide that specifically binds to PSG including, but not limited to, polyclonal, monoclonal, humanized, chimeric, single chain antibodies, fragments, or combinations thereof. The array optionally includes at least one second binding agent, wherein the second binding agent specifically binds to a second biomarker of an inflammatory pathology. Diagnosis of a vascular inflammatory pathologies can be accomplished by detecting the expression of PSG alone or in combination with at least one second biomarker of an inflammatory pathology.

Another aspect provides a method for diagnosing an inflammatory condition by determining the level of pregnancy-specific glycoprotein in a biological sample, preferably from a non-pregnant patient or host. The level of PSG is then compared with a predetermined value of PSG indicative of healthy vasculature. If the level of PSG of the patient or host is different from the predetermined value of PSG indicative of healthy vasculature, the patient or host is diagnosed with an inflammatory condition. An exemplary inflammatory condition is atherosclerosis.

A second biological marker indicative of an inflammatory condition can be assayed in combination with PSG levels. The diagnosing step can then be based on the level of the second biological marker and the level of PSG. Exemplary second biological markers include, but are not limited to, C-reactive protein, homocysteine, fibrinogen, lipoprotein, creatine kinase MB, troponin I, troponin T, creatine kinase, creatinine, fibrinogen, interleukin-1, PAPP-A, interleukin-6, a fragment or isoform thereof, and combinations thereof.

Still another aspect provides a method for treating an inflammatory condition by administering to a mammal in need thereof an amount of a PSG modulator effective to modulate PSG expression.

Yet another aspect provides a method for treating or preventing atherosclerosis by administering to a mammal in need thereof, a pharmaceutical composition effective to modulate the expression of PSG in vascular tissue.

### DETAILED DESCRIPTION

Generally, embodiments of the disclosure include methods and compositions for diagnosing, detecting, treating, and preventing vascular pathologies including, but not limited to, inflammatory conditions in a mammal (e.g., a human patient). Representative vascular pathologies include, but are not limited to, atherosclerosis, acute and chronic inflammatory conditions, and especially those inflammatory conditions as related to vasculature. Non-limiting examples of inflammatory conditions include acute coronary syndromes (unstable angina, acute myocardial infarction, sudden cardiac death, coronary plaque rupture, or thrombosis), Crohn's disease, vasculitis, Takayasu's arteritis, giant cell arterities, Kawasaki disease, inflammatory bowel disease, atherosclerosis and rheumatoid arthritis.

1. Definitions

Unless otherwise indicated the following terms used in the specification and claims have the meanings discussed below:

The term “PSG polypeptide” refers to a pregnancy-specific glycoprotein and variants, isoforms, or fragments thereof. PSG polypeptides include, but are not limited to pregnancy specific β1 glycoproteins 1-11 (SEQ ID Nos. 1-11).

The term “PSG nucleic acid” refers to polynucleotides encoding a PSG polypeptide or a complement thereof.

The term “organism” refers to any living entity comprised of at least one cell. A living organism can be as simple as, for example, a single eukaryotic cell or as complex as a mammal, including a human being.

The term “therapeutically effective amount” as used herein refers to that amount of the compound being administered which will relieve to some extent one or more of the symptoms of the disorder being treated. In reference to vascular pathologies or conditions, a therapeutically effective amount refers to that amount which has the effect of (1) reducing inflammation, plaque formation, or monocyte adhesion, (2) inhibiting (that is, slowing to some extent, preferably stopping) inflammation, plaque formation, or monocyte adhesion (3) relieving to some extent (or, preferably, eliminating) one or more symptoms associated with vascular inflammation including but not limited to atherosclerosis and other vascular inflammation pathologies.

“Pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, or a pharmaceutically acceptable salts thereof, with other chemical components, such as physiologically acceptable carriers and excipients. The purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to inflammation, these terms simply mean that the life expectancy of an individual affected with an inflammation pathology will be increased or that one or more of the symptoms of the disease will be reduced.

The term “prodrug” refers to an agent, including nucleic acids and proteins, which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS Pharm Sci., 2(1): E6; Sadzuka Y. (2000). Effective prodrug liposome and conversion to active metabolite, Curr Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

The term “nucleic acid” is a term of art that refers to a string of at least two base-sugar-phosphate combinations. For naked DNA delivery, a polynucleotide contains more than 120 monomeric units since it must be distinguished from an oligonucleotide. However, for purposes of delivering RNA, RNAi and siRNA, either single or double stranded, a polynucleotide contains 2 or more monomeric units. Nucleotides are the monomeric units of nucleic acid polymers. The term includes deoxyribonucleic acid (DNA) and ribonucleic acid (RNA). RNA may be in the form of an tRNA (transfer RNA), snRNA (small nuclear RNA), rRNA (ribosomal RNA), mRNA (messenger RNA), anti-sense RNA, RNAi, siRNA, and ribozymes. The term also includes PNAs (peptide nucleic acids), phosphorothioates, and other variants of the phosphate backbone of native nucleic acids. Anti-sense is a polynucleotide that interferes with the function of DNA and/or RNA. Natural nucleic acids have a phosphate backbone, artificial nucleic acids may contain other types of backbones, but contain the same bases.

The term “siRNA” means a small inhibitory ribonucleic acid. The siRNA are typically less than 30 nucleotides in length and can be single or double stranded. The ribonucleotides can be natural or artificial and can be chemically modified. Longer siRNAs can comprise cleavage sites that can be enzymatically or chemically cleaved to produce siRNAs having lengths less than 30 nucleotides, typically 21 to 23 nucleotides. siRNAs share sequence homology with corresponding target mRNAs. The sequence homology can be 100 percent or less but sufficient to result in sequence specific association between the siRNA and the targeted mRNA. Exemplary siRNAs do not activate the interferon signal transduction pathway.

The term “inhibitory nucleic acid” means an RNA, DNA, or combination thereof that interferes or interrupts the translation of mRNA. Inhibitory nucleic acids can be single or double stranded. The nucleotides of the inhibitory nucleic acid can be chemically modified, natural or artificial.

The term “sequence complementarity” means the degree of base-pairing (A opposite U or T, G opposite C) between two sequences of nucleic acids.

2. Description of Pregnancy-Specific β-1 Glycoproteins (PSGs)

Some embodiments of the present disclosure are directed to the detection and modulation of pregnancy-specific β-1 glycoproteins (PSGs). PSGs are a family of highly similar secreted proteins initially isolated from human placenta and pregnancy serum. PSGs comprise a subgroup of the carcinoembryonic antigen (CEA) family, which is composed of the PSG subfamily, the CEACAM subfamily and the CEACAM pseudogene (CEACAMP) subfamily. The members of the CEA/PSG gene family have a characteristic N-terminal domain that is homologous to the immunoglobulin variable region.

PSG genes encode at least 11 isoforms: PSG1, 2, 3, 4, 5, 6, 7, 8, 9, 10 and 11. For 7 out of 11 genes, two DNA sequences differing from each other in 1 to 4 nucleotides were detected. Most likely they represent different alleles. All of the PSGs except PSG1, PSG4, and PSG8 contain the arginine-glycine-aspartic acid sequence at position 93-95 corresponding to the complementary determining region 3 of immunoglobulin. At the protein level, PSGs isolated from human placenta includes a set of at least 3 glycoproteins with apparent molecular masses of 72, 64, and 54 kD, respectively. All PSGs appear to be secreted. PSG becomes detectable in serum during the first 2 to 3 weeks of pregnancy, and increases in concentration as pregnancy progresses, rising to a very high level of 200 to 400 micrograms per milliliter. Since low PSG levels are associated with poor pregnancy outcome [1], PSGs appear to be essential for maintenance of normal pregnancy. Elevated levels of PSGs are found in serum of patients with choriocarcinoma and hydatidiform mole [2,3]. The ectopic expression of PSGs in peripheral blood and bone marrow cells has been reported [4]. PSG has also been detected in testis tissue of males.

As described more fully below, the inventors have discovered that decreased PSG expression in vascular tissue is indicative of vascular pathology such as atherosclerosis.

3. Vascular Pathologies

Some embodiments of the present disclosure provide methods and compositions for detecting, diagnosing, treating or preventing vascular pathologies by detecting, measuring, or modulating the expression of PSGs in vascular tissue, for example vascular endothelial cells, vascular smooth muscle cells, or a combination thereof. Vascular pathologies include, but are not limited to, vascular inflammation, endothelial dysfunction, thrombosis, atherosclerosis, coronary artery disease, tachycardia, hypotension, hypertension, cerebrovascular disease, carotid artery bruits, focal neurological deficits, peripheral vascular disease, decreased peripheral pulses, peripheral arterial bruits, pallor, peripheral cyanosis, gangrene, ulceration, abdominal aortic aneurysm, pulsatile abdominal mass, peripheral embolism, circulatory collapse, and atheroembolism.

One embodiment is directed to the detection, diagnosis, or treatment of atherosclerosis, thrombosis, or restenosis by detecting or modulating PSG expression in vascular tissue. Particularly effective compositions for preventing vascular pathologies are those that increase PSG expression in vascular tissue. Ideal compositions increase the expression of PSG in vascular tissues without affecting PSG expression in other tissues. Atherosclerosis is generally known as a disease of the blood vessels, for example the arteries. More particularly, atherosclerosis generally affects large and medium-sized muscular arteries, but it will be appreciated by one of skill in the art, that atherosclerosis can include any size blood vessel. Generally, atherosclerosis includes endothelial dysfunction, vascular inflammation, and the buildup of lipids, cholesterol, calcium, and cellular debris within the intima of the vessel wall. This buildup results in plaque formation, vascular remodeling, acute and chronic luminal obstruction, abnormalities of blood flow, and diminished oxygen supply to target organs.

The specific mechanism of atherosclerosis is unclear; however, one commonly accepted theory is the “response-to-injury” theory. Under this theory, endothelial injury causes vascular inflammation and a fibroproliferative response ensues. Exemplary causes of endothelial injury include, but are not limited to, oxidized low-density lipoprotein (oxLDL) cholesterol; infectious agents; toxins, including the byproducts of cigarette smoking; hyperglycemia; and hyperhomocystinemia. Circulating monocytes infiltrate the intima of the vessel wall, and these tissue macrophages act as scavenger cells, taking up LDL cholesterol and forming the characteristic foam cell of early atherosclerosis. These activated macrophages produce numerous factors that are injurious to the endothelium.

Platelets adhere to the area of endothelial disruption and release a number of growth factors, including platelet derived growth factor (PDGF). PDGF, which is also released by foam cells and altered endothelial cells, stimulates migration and proliferation of vascular smooth muscle cells into the lesion. These smooth muscle cells release extracellular matrix (collagen and elastin) and the lesion continues to expand. Macrophages in the lesion secrete proteases, and the resulting cell damage creates a necrotic core filled with cellular debris and lipid. The lesion is then referred to as a “complex lesion.” Rupture of this lesion can lead to thrombosis and occlusion of the blood vessel. In the case of a coronary artery, rupture of a complex lesion may precipitate a myocardial infarction, whereas in the case of a carotid artery, stroke may ensue.

Balloon angioplasty is one method used to reopen a blood vessel which is narrowed by plaque. Although balloon angioplasty is successful in a high percentage of the cases in opening the vessel, it unfortunately denudes the endothelium and injures the vessel in the process. This damage causes the migration and proliferation of vascular smooth muscle cells of the blood vessel into the area of injury to form a lesion, known as myointimal hyperplasia or restenosis. This new lesion leads to a recurrence of symptoms within three to six months after the angioplasty in a significant proportion of patients (30-40%).

In atherosclerosis, thrombosis and restenosis there is also a loss of normal vascular function, such that vessels tend to constrict, rather than dilate. The excessive vasoconstriction of the vessel causes further narrowing of the vessel lumen, limiting blood flow. This can cause symptoms such as angina (if a heart artery is involved), or transient cerebral ischemia (i.e. a “small stroke”, if a brain vessel is involved). This abnormal vascular function (excessive vasoconstriction or inadequate vasodilation) occurs in other disease states as well. Hypertension (high blood pressure) is caused by excessive vasoconstriction, as well as thickening, of the vessel wall, particularly in the smaller vessels of the circulation. This process may affect the lung vessels as well causing pulmonary (lung) hypertension. Other disorders known to be associated with excessive vasoconstriction, or inadequate vasodilation include transplant atherosclerosis, congestive heart failure, toxemia of pregnancy, Raynaud's phenomenon, Prinzmetal's angina (coronary vasospasm), cerebral vasospasm, hemolytic-uremia and impotence.

4. Diagnosing Vascular Pathologies

4.1 In Vitro Diagnosis

Another embodiment provides a method for diagnosing vascular pathologies such as an inflammatory condition in a mammal, in particular a non-pregnant mammal. An exemplary method includes measuring the level of pregnancy-specific glycoprotein (PSG) in a biological sample from a non-pregnant patient and comparing the level with a predetermined level of PSG indicative of healthy vascular tissue. The predetermined level of PSG can be obtained from one or more control subjects that do not have a vascular pathology, for example, a mammal such as a human that does not have atherosclerosis. The method also includes diagnosing the inflammatory condition based on the level of PSG of the non-pregnant mammal relative to that of control subjects or a predetermined level, wherein the patient is diagnosed as having inflammatory condition if the level of PGS is decreased relative to that of control subjects. Without wishing to be bound by any one theory, it is believed that PGS expression reduces or prevents vascular pathologies such as atherosclerosis. Accordingly, an elevated level of detectable PSG compared to a control sample can be indicative of healthy vascular tissue and a lower level of detectable PSG compared to a control sample can be indicative of a vascular pathology.

Representative biological samples include, but are not limited to biological fluids and tissues. Biological fluids include, but are not limited to whole blood, plasma, serum, tears, saliva, urine, sweat, and semen. Biological tissues include vascular tissues as wells as individual cells of an organism.

4.1.1 Detection of PSG

PSG or a fragment of PSG can be detected in a mammal or biological fluid using conventional immunotechniques. For example, a sandwich assay can be performed by capturing PSG or a fragment thereof from a biological sample with an antibody having specific binding affinity for PSG. PSG then can be detected with a labeled antibody having specific binding affinity for PSG. Alternatively, standard immunohistochemical techniques can be used to detect PSG protein or an isomer thereof, using such antibodies.

The production of antibodies is well known in the art. Briefly, various host animals can be immunized by injection of one or more isoforms of PSG. Host animals include rabbits, chickens, mice, guinea pigs, horses, swine, and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin and dinitrophenol. Polyclonal antibodies are heterogenous populations of antibody molecules that are contained in the sera of the immunized animals. Monoclonal antibodies, which are homogeneous populations of antibodies to a particular antigen, can be prepared using a PSG polypeptide and standard hybridoma technology. In particular, monoclonal antibodies can be obtained by any technique that provides for the production of antibody molecules by continuous cell lines in culture such as described by Kohler, G. et al., Nature, 256:495 (1975), the human B-cell hybridoma technique (Kosbor et al., Immunology Today, 4:72 (1983); Cole et al., Proc. Natl. Acad. Sci USA, 80:2026 (1983)), and the EBV-hybridoma technique (Cole et al., “Monoclonal Antibodies and Cancer Therapy”, Alan R. Liss, Inc., pp. 77-96 (1983)). Such antibodies can be of any immunoglobulin class including IgG, IgM, IgE, IgA, IgD, and any subclass thereof. The hybridoma producing the monoclonal antibodies of the invention can be cultivated in vitro and in vivo.

Antibody fragments that have specific binding affinity for PSG polypeptide can be generated by known techniques. For example, such fragments include but are not limited to F(ab′)2 fragments that can be produced by pepsin digestion of the antibody molecule, and Fab fragments that can be generated by reducing the disulfide bridges of F(ab′)2 fragments. Alternatively, Fab expression libraries can be constructed. See, for example, Huse et al., Science, 246:1275 (1989). Once produced, antibodies or fragments thereof are tested for recognition of PSG by standard immunoassay methods including ELISA techniques, radioimmunoassays and Western blotting. See, Short Protocols in Molecular Biology, Chapter 11, Green Publishing Associates and John Wiley & Sons, Edited by Ausubel, F. M. et al., 1992. Antibodies having affinity for PSG are identified in a positive selection.

It will be appreciated by those of skill in the art that the disclosed methods can include the detection of specific PSG nucleic acids, for example PSG mRNA in the biological sample. Techniques for the rapid detection of nucleic acids are known in the art. PSG message can be detected, for example, by a polymerase chain reaction (PCR) assay. In general, PCR refers to amplification of a target nucleic acid, using sequence information from the ends of the region of interest or beyond to design oligonucleotide primers that are identical or similar in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. PCR is described, for example in PCR Primer: A Laboratory Manual, Ed. by Dieffenbach, C. and Dveksler, G., Cold Spring Harbor Laboratory Press, 1995. Nucleic acids also can be amplified by ligase chain reaction, strand displacement amplification, self-sustained sequence replication or nucleic acid sequence-based amplification. See, for example, Lewis, R., Genetic Engineering News, 12(9):1 (1992); Guatelli et al., Proc. Natl. Acad. Sci. USA, 87:1874-1878 (1990); and Weiss, R., Science, 254:1292 (1991).

The levels of PSG mRNA can be detected using reverse transcription-polymerase chain reaction (RT-PCR) assay. See, for example, WO 00/54806. In particular, PSG cDNA can be coamplified with a deletion variant thereof that is used as an internal standard (IS). The amount of PSG is normalized against the total amount of mRNA in the sample, determined as the amount of β-actin mRNA. RT-PCR has been shown to be 1,000-10,000 fold more sensitive than traditional RNA blotting techniques, and can detect and quantitate PSG mRNA in tissue samples.

Products from competitive PCR can be quantified by ion exchange chromatography on an HPLC system, an accurate method that involves a minimum of post-PCR handling. Alternatively, real-time quantitative PCR can be performed using, for example, the ABI PRISM 7700 Sequence Detection System and Taqman fluorogenic probes, or the LightCycler™ instrument from Roche. An internal reference can be used, such as amplification of the 28S rRNA with limiting primer concentration. This method allows quantitation down to approximately 500 copies of the target sequence.

Alternatively, testing different tissues for the presence of specific mRNAs can be done routinely by RNA blotting techniques such as Northern or dot blotting or through microarray technology.

Levels of PSG can be determined using microarrays (see Section 5). For example, protein levels of PSG can be detected using a protein-antibody array or a proteomic array. The protein array can also include markers for other biomarkers for vascular pathology. Alternatively, DNA microarrays can be used to detect PSG nucliec acids, such as mRNA or DNA for PSG. The DNA microarrays can also detect one or more other biomarkers for vascular pathology.

4.1.2 Combination of Biomarkers for Diagnosis

The diagnosis of a vascular pathology can be made using detectable levels of PSG in a biological sample from a patient in combination with levels of other biomarkers indicative of vascular pathology. For example, low levels of detectable PSG in a non-pregnant mammal combined with elevated levels of a biological marker indicative of vascular pathology such as C-reactive protein may be considered together for the diagnosis of a vascular pathology such as atherosclerosis. Biological markers that may be useful to measure in combination with PSG include lipid profiles and polypeptide markers of inflammation, markers correlating with increased risk of atherosclerosis, unstable angina or myocardial infarction (e.g., homocysteine), markers of cardiac injury, and other non-specific markers of inflammation. Representative biological markers for atherosclerosis include, but are not limted to C-reactive protein levels, homocysteine levels, fibrinogen levels, and lipoprotein levels. Additionally, interleukin-1 (IL-1), IL-6, or neopterin can be assessed in combination with PSG as a marker for inflammation. Cardiac markers and non-specific markers of inflammation include, for example, troponin I or T, C-reactive protein, creatine kinase (CK), CK-MB, creatinine, myoglobin, and fibrinogen.

Particular combinations of polypeptides that can be used for diagnosing a patient with acute coronary syndrome include, for example, PSG, troponin I, and CK-MB; PSG, troponin I, and C-reactive protein; PSG, CK-MB, and myoglobin; PSG and myoglobin; PSG and C-reactive protein PSG and troponin I or T; and PSG and CK-MB. In general, myoglobin is not cardiac specific, but is released from infarcted myocardium at an early stage (about 2-3 hours post infarction) and returns to normal within about 24 hours. Cardiac isoforms of troponin I and troponin T are specific, but appear in the circulation later than myoglobin (5 to 48 hours post infarction). Myocardial tissue contains one isoform of CK-MB, while skeletal tissue has different isoforms. Antibodies having specific binding affinity for such cardiac markers are available commercially.

4.2 Visualization of PSG In Vivo

Inflammatory conditions also can be diagnosed by administering an amount of a label, for example an antibody, having specific binding affinity for PSG to a patient in an amount effective for visualizing PSG in vivo. The label can be any detectable substance that specifically binds to PSG, a fragment of PSG, or to a complex containing PSG or a fragment of PSG. In addition, visualizing PSG would allow sites in the body with normal vasculature to be identified. The absence of detectable PSG in vascular tissue can be indicative of a vascular pathology.

Suitable antibodies and methods for making antibodies are known in the art. The antibody typically is labeled, and diagnostic imaging is used to detect antibody bound to PSG. Diagnosis of the inflammatory condition is based on the increase or decrease of PSG compared to a control sample having a PSG levels indicative of healthy vascular without inflammation. A threshold can be set to any level, so a level over normal can be detected. Thus, diagnosis can be made based on the presence or absence of antibody bound to PSG.

Typical labels that are useful include metal particles typically less than 100 nm in diameter, fluorophores such as infrared fluorophores, radioisotopes used for imaging procedures in humans, and any other detectable label known in the art. Non-limiting examples of labels include radioisotope such as ¹²³I (iodine), ¹⁸F (fluorine), ⁹⁹Tc (technetium), ¹¹¹In (indium), and ⁶⁷Ga (gallium). Antibodies can be labeled through standard techniques. For example, antibodies can be iodinated using chloramine T or 1,3,4,6-tetrachloro-3α,6α-diphenylglycouril. Antibodies can be labeled with ¹⁸F through, for example, N-succinimidyl 4-[¹⁸F]fluorobenzoate. See, Muller-Gartner, H., TIB Tech., 16:122-130 (1998); Saji, H., Crit. Rev. Ther. Drug Carrier Syst., 16(2):209-244 (1999); and Vaidyanathan and Zalutsky, Bioconjug. Chem. 5(4):352-6 (1994) for a review of labeling of antibodies with such radioisotopes.

The labeled antibodies can be formulated with a pharmaceutically acceptable carrier and administered to the patient. In general, the antibodies are administered intravenously (i.v.), although other parenteral routes of administration, including subcutaneous, intramuscular, intrarterial, intracarotid, and intrathecal also can be used. Formulations for parenteral administration may contain pharmaceutically acceptable carriers such as sterile water or saline, polyalkylene glycols such as polyethylene glycol, vegetable oils, hydrogenated naphthalenes, and the like.

The dosage of labeled antibody to be administered will be determined by the attending physician taking into account various factors known to modify the action of drugs. These include health status, body weight, sex, diet, time and route of administration, other medications, and any other relevant clinical factors.

Imaging techniques that can be used to detect PSG in vivo include positron emission tomography (PET), gamma-scintigraphy, magnetic resonance imaging (MRI), functional magnetic resonance imaging (FMRI), single photon emission computerized tomography (SPECT), and intravascular ultrasound.

5. Kits and Arrays for Diagnosing Inflammatory Conditions

Antibodies having specific binding affinity for PSG can be combined with packaging material and sold as a kit for diagnosing inflammatory conditions. Components and methods for producing kits are well known. The kits may combine one or more anti-PSG antibodies or fragments thereof as described herein. In addition, the kits may further include reagents for measuring levels of a plurality of polypeptides in a biological sample, including, for example, antibodies having specific binding affinity to the particular polypeptide, secondary antibodies, indicator molecules, solid phases (e.g., beads) and/or other useful agents for diagnosing inflammatory conditions. Instructions describing how the various reagents are effective for diagnosing inflammatory conditions also may be included in such kits. Biological markers that may be useful to measure in combination with PSG include lipid profiles and polypeptide markers of inflammation, markers correlating with increased risk of atherosclerosis, unstable angina or myocardial infarction (e.g., homocysteine), markers of cardiac injury, and other non-specific markers of inflammation. Representative biological markers for atherosclerosis include, but are not limited to C-reactive protein levels, homocysteine levels, PAPP-A, fibrinogen levels, and lipoprotein levels. Additionally, interleukin-1 (IL-1), IL-6, or neopterin can be assessed in combination with PSG as a marker for inflammation. Cardiac markers and non-specific markers of inflammation include, for example, troponin I or T, C-reactive protein, creatine kinase (CK), CK-MB, creatinine, myoglobin, and fibrinogen.

Particular combinations of polypeptides that can be used for diagnosing a patient with acute coronary syndrome include, for example, PSG, troponin I, and CK-MB; PSG, troponin I, and C-reactive protein; PSG, CK-MB, and myoglobin; PSG and myoglobin; PSG and C-reactive protein; PSG and troponin I or T; and PSG and CK-MB. In general, myoglobin is not cardiac specific, but is released from infarcted myocardium at an early stage (about 2-3 hours post infarction) and returns to normal within about 24 hours. Cardiac isoforms of troponin I and troponin T are specific, but appear in the circulation later than myoglobin (5 to 48 hours post infarction). Myocardial tissue contains one isoform of CK-MB, while skeletal tissue has different isoforms. Antibodies having specific binding affinity for such cardiac markers are available commercially.

The anti-PSG antibody can be in a container, such as a plastic, polyethylene, polypropylene, ethylene, or propylene vessel that is either a capped tube or a bottle. Non-limiting examples of other reagents that can be included in the kit are, for example, labeled, secondary antibodies that bind to the anti-PSG antibody and buffers for washing or detecting PSG. Reagents for measuring levels of other polypeptides can be included in separate containers or can be included on a solid phase with anti-PSG antibody, e.g., a handheld device for bedside testing that includes anti-PSG antibody and one or more antibodies having specific binding affinity for markers of inflammation or in particular, cardiac injury.

Another embodiment of the present disclosure provides arrays for diagnosing a vascular pathology in host. One embodiment provides an array for diagnosing a vascular pathology, for example atherosclerosis. The disclosed arrays are capable of measuring or detecting multiple markers of vascular pathologies at the same time. Detecting multiple markers of vascular pathologies increases the accuracy of diagnosis and prognosis of the diseases. One embodiment provides an array having a body portion with a binding agent specific for PSG, for example nucleic acids complementary to PSG attached thereto. The nucleic acids are typically about 6 to about 60 nucleotides, more typically about 8 to about 20 but it will be appreciated that any size will do so long as the nucleic acids specifically hybridize to their target PSG nucleotide sequence, typically an RNA sequence. A sample can be obtained from a patient and applied to the array. Binding of a PSG polypeptide or PSG nucleic acid, isoforms, or variants thereof from the sample to the array in an amount less than a predetermined level is indicative of a vasculator pathology. The predetermined level can be determined by assaying the levels of PSG polypeptides or PSG nucleic acids in patients or hosts without vascular pathology.

Methods for producing and using microarrays are known in the art. Briefly, microarrays are constructed by arraying PCR amplified cDNA clones or genes at high density on an insoluble substrate, for example, derivatized glass microscope slides, metal surfaces, or polymer surfaces such as plastic surfaces. Generally, cDNA clones of biological markers for vascular pathologies such as atherosclerosis are produced and fixed to a surface of the array. The biological markers include PSG, optionally in combination with at least one second biological marker of vascular pathology, including, but not limited to, VCAM, ICAM, integrins, cell surface proteins, C-reactive protein, PAPP-A, fibrinogen levels, lipoprotein levels, interleukin-1 (IL-1), IL-6, or neopterin or cell adhesion molecules.

Microarrays can be prepared by printing PCR amplicons suspended in either a high salt or other denaturing buffer onto poly-L-lysine or aminosilane coated glass microscope slides, for example using a high-speed robotic system such as is commercially available. The arrayer can use a 12-tip print head to array DNA samples from either 96- or 384-well microtiter plates onto as many as 100 silanized glass microscope slides. With an average spot size of about 130 μm and the capability to adjust the spot-to-spot spacing, the arrayer can spot 19,200 elements (the contents of 200 microtiter plates) or more onto a single slide.

Aminosilane coated glass microscope slides can offer a more consistent surface with lower background fluorescence. Appropriate buffers for printing the arrays can be used. For example 50% DMSO is a representative printing buffer. Using 50% DMSO as a printing solution has a number of additional advantages. DMSO denatures the DNA allowing better binding to the slide and providing more single-stranded targets for hybridization. Further, DMSO is hygroscopic and has a low vapor pressure, allowing DNA prepared for arraying to be stored for long periods of time without significant evaporation.

The print head on the arrayer can use “quill” pens that use capillary action to draw fluid into the spotting pens and surface tension interactions to dispense solution onto the slide. A variety of parameters such as the robot arm acceleration, temperature, and humidity control both spot morphology and size. Suitable conditions include approximately 45% relative humidity and a constant temperature of 72° F.

Probes for microarray analysis can be prepared from RNA templates by incorporation of labeled deoxyribonucleotides during first strand cDNA synthesis. Representative labels include, but are not limited to, fluorophores, radioisotopes, nanoparticles, metal particles, and the like. The RNA templates are obtained from a patient or host, typically from a host's vascular tissue. Either total or poly(A+) RNA can be used in the reverse transcription reaction. Oligo(dT) labeling of total RNA provides consistently high-quality probes from smaller quantities of starting RNA and without the expense of poly(A+) purification.

Aminosilane coated slides bind DNA with high efficiency. Prior to hybridization, the free amine groups on the slide should be blocked or inactivated, otherwise nonspecific binding of labeled cDNA to the slide can deplete the probe and produce high background. Although the slides can be blocked chemically, prehybridization in a solution containing 1% bovine serum albumin can reduce nonspecific binding of the probe to the slide.

Prehybridization has the additional advantage of washing unbound DNA from the slide prior to the addition of the probe. Any DNA that washes from the surface during hybridization competes with DNA bound to the slide. As the kinetics of solution hybridization is much more favorable than surface hybridization, this can dramatically decrease the measured fluorescence signal from the microarray. Differential gene expression is assessed by scanning the hybridized arrays and detecting the labeled RNA for example using a confocal laser scanner.

Another embodiment provides a kit for diagnosing a vascular pathology such as atherosclerosis. The kit includes a microarray capable of detecting PSG levels and optionally, at least one second biological marker for vascular pathology. Instructions for using the microarray as wells a buffers and other reagents can also be included.

Another embodiment provides an array, for example an antibody array, having a body portion with polypeptides attached to a surface thereof. The polypeptides specifically bind to PSG, isomers, or fragments thereof. Suitable polypeptides include, but are not limited to, antibodies specific for PSG. The antibodies can be polyclonal, monoclonal, fragments, single chain antibodies, humanized, or chimeric antibodies. Methods for producing such antibodies are known in the art. The antibodies can be specific for at least one epitope of a PSG polypeptide or fragment thereof. The antibody arrays can optionally include polypeptides that bind to other polypeptides known or suspected to be involved in vascular pathologies including, C-reactive protein, PAPP-A, fibrinogen, lipoprotein, interleukin-1 (IL-1), IL-6, or neopterin.

A representative antibody microarray includes a plurality of antibodies bound in an ordered layout to a glass slide. The antibodies can be covalently bound or releasably bound to a surface of the microarray. Generally, polypeptides are obtained from a patient or host, in particular from vascular tissue or vascular cells. The polypeptides obtained from the host are then labeled with a detectable label, for example a fluorophore, biotin-strepavidin, an enzyme, or radioisotope using conventional labeling protocols. The labeled polypeptides are placed on the array under conditions that favor hybridization, for example physiological conditions. Representative physiological conditions include 37° C. and neutral pH. Excess labeled polypeptide is washed off the array, and the array is scanned with a device capable of detecting the labeled polypeptides that have hybridized to the array.

6. Screening For Modulators of the Protein Function or Expression

Embodiments of the present disclosure include methods for identifying modulators of the function, expression, or bioavailability of PSGs in vascular tissue. The modulator may modulate one or more specific PSGs directly or indirectly. Direct modulation refers to a physical interaction between the modulator and the PSG, for example binding of the modulator to a region of the PSG. Indirect modulation of the PSG can be accomplished when the modulator physically associates with a cofactor, second protein or second biological molecule that interacts with the PSG either directly or indirectly. Additionally, indirect modulation would include modulators that affect the expression of PSG RNA or the translation of PSG RNA.

In some embodiments, the assays can include random screening of large libraries of test compounds. Alternatively, the assays may be used to focus on particular classes of compounds suspected of modulating the function or expression of PSGs in vascular tissue as a result of the classes of compounds containing a specific structure or motif.

Assays can include determinations of protein expression, protein activity, or binding activity. Other assays can include determinations of nucleic acid transcription or translation, for example mRNA levels, mRNA stability, mRNA degradation, transcription rates, and translation rates.

In one embodiment, the identification of a PSG modulator is based on the function of the PSG in the presence and absence of a test compound. The test compound or modulator can be any substance that alters or is believed to alter the function of a PSG, in particular the function of PSG in vascular tissue. One exemplary method includes obtaining a PSG, contacting the PSG with at least a first test compound, and assaying for an interaction between the PSG and the first test compound with an assay. The assaying can include determining PSG induction of the expression of nucleic acids in vascular tissue, including for example, expression of an adhesion molecule, a receptor, a signaling molecule, a cytokine or an enzyme in vascular tissue.

Specific assay endpoints or interactions that may be measured in the disclosed embodiments include, but are not limited to, assaying for inducible nitric oxide synthase (iNOS) induction, receptor for advanced glycation or glycosylation Double check the difference between glycosylation endproducts, monocyte chemoattractant protein-1, P-selectin, endothelin-1, endothelin-receptor, interleukin-6 or heme oxygenase-1. These assay endpoints may be assayed using standard methods such as FACS, ELISA, Northern blotting and/or Western blotting. Moreover, the assays can be conducted in cell free systems, in isolated cells such as vascular tissue cells, genetically engineered cells, immortalized cells, or in organisms including transgenic animals.

Other screening methods include using labeled PSG to identify a test compound. PSG can be labeled using standard labeling procedures that are well known and used in the art. Such labels include, but are not limited to radioactive, fluorescent, biological and enzymatic tags.

Another embodiment provides a method for identifying a modulator of PSG expression by detemining the effect a test compound has on the expression of PSG in vascular tissue cells such as vascular smooth muscle cells or vascular endothelial cells. For example, a vascular cell or any cell expressing PSG can be contacted with a test compound. PSG expression can be determined by detecting PSG protein expression or PSG mRNA transcription or translation. Suitable cells for this assay include, but are not limited to, immortalized cell lines, primary cell culture, or cells engineered to express PSG. Compounds that modulate the expression of PSG, in particular that increase the expression of PSG, can be selected as therapeutic agents for the treatment of athersclerosis.

6.1 Modulators

As used herein the term “test compound” or “modulator” refers to any molecule that may potentially inhibit or enhance PSG activity or expression, in particular PSG activity or expression in vascular tissue or vascular cells. Preferred modulators increase PSG activity or expression in vascular tissue. Representative modulators include, but are not limited to, TNFα, IL1β, TGFβ and PDGF. The test compound can be a protein or fragment thereof, a small molecule, or even a nucleic acid molecule. Some test compounds can be compounds that are structurally related to PSG, anti-inflammatory molecules, or pro-inflammatory molecules, i.e., adhesion molecules, surface receptors, cytokines, or other substances induced or repressed by PSG. Using lead compounds to help develop improved compounds is known as “rational drug design” and includes not only comparisons with known inhibitors and activators, but predictions relating to the structure of target molecules.

Alternatively, small molecule libraries that are believed to meet the basic criteria for useful drugs can be screened to identify useful compounds. Screening of such libraries, including combinatorially generated libraries (e.g., expression libraries), is a rapid and efficient way to screen large a number of related (and unrelated) compounds for activity. Combinatorial approaches also lend themselves to rapid evolution of potential drugs by the creation of second, third and fourth generation compounds modeled of active, but otherwise undesirable compounds.

Test compounds may include fragments or parts of naturally-occurring compounds, or may be found as active combinations of known compounds, which are otherwise inactive. Compounds isolated from natural sources, such as animals, bacteria, fungi, plant sources, including leaves and bark, and marine samples can be assayed as candidates for the presence of potentially useful pharmaceutical agents. It will be understood that the pharmaceutical agents to be screened could also be derived or synthesized from chemical compositions or man-made compounds. Thus, it is understood that the test compound identified by embodiments of the present disclosure may be peptide, polypeptide, polynucleotide, small molecule inhibitors, small molecule inducers, organic or inorganic, or any other compounds that may be designed based on known inhibitors or stimulators.

Other suitable modulators include antisense molecules, catalytic nucleic acids such as ribozymes, and antibodies (including single chain antibodies), each of which would be specific for one or more PSGs. For example, an antisense molecule that binds to a translational or transcriptional start site, or splice junctions, is within the scope of a test compound.

In addition to the modulating compounds initially identified, other sterically similar compounds may be formulated to mimic the key portions of the structure of the modulators. Such compounds, which may include peptidomimetics of peptide modulators, may be used in the same manner as the initial modulators.

An inhibitor or activator according to the present disclosure may be one which exerts its inhibitory or activating effect upstream, downstream, directly, or indirectly on one or more PSGs. In one embodiment, the inhibition or activation by an identified modulator results in the modulation of PSG biological activity or expression as compared to that observed in the absence of the added test compound.

6.2 In Vitro Assays

Another embodiment provides for in vitro assays for the identification of PSG modulators. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule, for example a PSG nucleic acid, in a specific fashion is strong evidence of a related biological effect. Such a molecule can bind to a PSG nucleic acid and modulate expression of PSG, for example upregulate expression of PSG. The binding of a molecule to a target may, in and of itself, be inhibitory, due to steric, allosteric or charge—charge interactions or may upregulate or activate PSG. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

6.3 Cell Assays

Other embodiments include methods of screening compounds for their ability to modulate PSG in cells. Various cell lines can be utilized for such screening assays, including cells specifically engineered for this purpose. Suitable cells include, but are not limited to, mammalian endothelial or smooth muscle cells from saphenous vein (SVEC) or coronary artery (CAEC). Cells can also be engineered to express PSG or a modulator of PSG or a combination of both PSG and a modulator of PSG. Furthermore, those of skill in the art will appreciate that stable or transient transfections, which are well known and used in the art, may be used in the disclosed embodiments.

For example, a transgenic cell comprising an expression vector can be generated by introducing the expression vector into the cell. The introduction of DNA into a cell or a host cell is well known technology in the field of molecular biology and is described, for example, in Sambrook et al., Molecular Cloning 3rd Ed. (2001). Methods of transfection of cells include calcium phosphate precipitation, liposome mediated transfection, DEAE dextran mediated transfection, electroporation, ballistic bombardment, and the like. Alternatively, cells may be simply transfected with the disclosed expression vector using conventional technology described in the references and examples provided herein. The host cell can be a prokaryotic or eukaryotic cell, or any transformable organism that is capable of replicating a vector and/or expressing a heterologous gene encoded by the vector. Numerous cell lines and cultures are available for use as a host cell, and they can be obtained through the American Type Culture Collection (ATCC), which is an organization that serves as an archive for living cultures and genetic materials (www.atcc.org).

A host cell can be selected depending on the nature of the transfection vector and the purpose of the transfection. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE, La Jolla, Calif.). Alternatively, bacterial cells such as E. coli LE392 could be used as host cells for phage viruses. Eukaryotic cells that can be used as host cells include, but are not limited to yeast, insects, plants, and mammals. Examples of mammalian eukaryotic host cells for replication and/or expression of a vector include, but are not limited to, HeLa, NIH3T3, Jurkat, 293, Cos, CHO, Saos, and PC12. Examples of yeast strains include, but are not limited to, YPH499, YPH500 and YPH501. Many host cells from various cell types and organisms are available and would be known to one of skill in the art. Similarly, a viral vector may be used in conjunction with either an eukaryotic or prokaryotic host cell, particularly one that is permissive for replication or expression of the vector.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

6.4 In Vivo Assays

In vivo assays involve the use of various animal models, including non-human transgenic animals that have been engineered to have specific defects or carry markers that can be used to measure the ability of a test compound to reach and affect different cells within the organism. Due to their size, ease of handling, and information on their physiology and genetic make-up, mice are a preferred embodiment, especially for transgenic animals. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more test compounds are administered to an animal, and the ability of the test compound(s) to alter one or more characteristics, as compared to a similar animal not treated with the test compound(s), identifies a modulator. The characteristics may be any of those discussed above with regard to the function of a particular compound (e.g., enzyme, receptor, hormone) or cell (e.g., growth, tumorigenicity, survival), or instead a broader indication such as angina, myocardial infarction, atherosclerosis, etc.

Other embodiments provide methods of screening for a test compound that modulates the function of PSG. In these embodiments, a representative method generally includes the steps of administering a test compound to the animal and determining the ability of the test compound to reduce one or more characteristics of vascular inflammation or atherosclerosis.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

7. Combination Therapy

Compositions that modulate the expression of PSGs in vascular tissue can be used in combination with a second therapeutic agent. Therapeutic agents for the treatment of vascular pathologies are known in the art and include, but are not limited to HMG-CoA reductase inhibitors, fibric acid derivatives, bile acid sequestrants, antioxidants, and nicotinic acid derivatives.

Exemplary HMG-CoA reductase inhibitors include, but are not limited to, pravastatin, simvastatin, lovastatin, fluvastatin, atorvastatin, and rosuvatstatin. Exemplary fibric acid derivatives include, but are not limited to, fenofibrate and gemfibrozil. Suitable bile sequenstrants include, but are not limited to, cholestyramine, and colestipol. Representative antioxidants include, but are not limited to, vitamin E and vitamin C. Suitable nicotinic acid derivatives include, but are not limited to, niacin.

8. Transgenic Animals/Knockout or Knockdown Animals

In one embodiment, transgenic animals are produced which contain a functional transgene encoding a functional PSG or modulator of PSG or a modified modulator of PSG involved in vascular inflammation. Transgenic animals expressing transgenes of PSG or a modulator or modified modulator of PSG involved in vascular inflammation, recombinant cell lines derived from such animals and transgenic embryos may be useful in methods for screening for and identifying agents that induce or repress function of PSG in vascular tissue or cells. Such transgenic animals can be used as models for studying disease states such as atherosclerosis.

One embodiment includes introducing a transgene into a non-human host to produce a transgenic animal expressing an exogenous nucleic acid, such as a human or murine gene. The transgenic animal is produced by the integration of the transgene into the genome in a manner that permits the expression of the transgene. Methods for producing transgenic animals are known in the art.

In other embodiments, the endogenous PSG or modulator of PSG can be replaced by homologous recombination between the transgene and the endogenous gene; or the endogenous gene may be eliminated by deletion as in the preparation of “knock-out” animals. Typically, the transgene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. In one embodiment, transgenic mice are generated which overexpress PSG in vascular tissue. Alternatively, the absence of PSG in “knock-out” or “knock down” mice permits the study of the effects of loss of PSG in vascular tissue. Yet further, the test compound may be overexpressed or “knocked-out” to further study the interaction of the test compound with PSG in vascular tissue.

As noted above, transgenic animals and cell lines derived from such animals may find use in certain testing experiments. In this regard, transgenic animals and cell lines capable of expressing PSG may be exposed to test compounds. These test substances can be screened for the ability to enhance or inhibit one or more characteristics of PSG, such as, expression of adhesion molecules, receptors, cytokines, signaling molecules or enzymes.

9. Uses of the PSG Modulators

Still other embodiments provide several uses for modulators of PSG, for example in vascular tissue. One embodiment provides administering the disclosed PSG modulators to a subject, such as a mammal, in an effective amount to modulate PSG expression, in particular to modulate PSG expression in vascular smooth muscle cells, endothelial cells, or a combination thereof to treat a vascular pathology such as atherosclerosis. The disclosed modulators may be administered to a subject with atherosclerosis, vascular inflammation, unstable angina, or acute myocardial infarction. It is also contemplated that these compositions reduce or alleviate symptoms related to vascular inflammation, for example, resulting in decreased atherosclerosis, decreased local inflammatory response, and decreased myocardial infarction. The modulator may inhibit the development of atherosclerosis, a stroke or other inflammatory diseases, e.g., rheumatoid arthritis, lupus and inflammatory bowel disease.

The modulator may be administered to a subject in a single dose or a series of doses. The series of doses may be administered daily, weekly, monthly, annually, or whenever it is deemed necessary. Specifically, the modulator may be administered during or prior to an anticipated “flare-up” or “acute episode” or “exacerbation” of the disease.

Another embodiment provides administering a pharmaceutical composition to a mammal in need thereof in an amount effective to increase PSG expression, for example in vascular tissue, vascular smooth muscle cells, endothelial cells, or a combination thereof. The pharmaceutical composition can contain a PSG modulator as an active ingredient. The PSG modulator can comprise a growth factor. Representative PSG modulators include, but are not limited to, TNFα, TGFβ, PDGF, IL1β, a fragment thereof, and combinations thereof.

10. Isolation of a Modulator

In some embodiments, the test compound or PSG modulator may be isolated and/or purified using standard procedures well known in the art. A test compound or PSG modulator may be a protein, peptide, polysaccharide, monosaccharide, carbohydrate, a small molecule, or a nucleic acid sequence. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Any of a wide variety of chromatographic procedures may be employed to isolate and/or purify the test compound or modulator that is a small molecule. For example, thin layer chromatography, gas chromatography, high performance liquid chromatography, paper chromatography, affinity chromatography or supercritical flow chromatography may be used to effect separation of various chemical species.

Partition chromatography is based on the theory that if two phases are in contact with one another, and if one or both phases constitute a solute, the solute will distribute itself between the two phases. Usually, partition chromatography employs a column, which is filled with a sorbent and a solvent. The solution containing the solute is layered on top of the column. The solvent is then passed through the column, continuously, which permits movement of the solute through the column material. The solute can then be collected based on its movement rate. The two most common types of partition chromatography are paper chromatography and thin-layer chromatography (TLC); together these are called adsorption chromatography. In both cases, the matrix contains a bound liquid. Other examples of partition chromatography are gas-liquid and gel chromatography.

Paper chromatography is a variant of partition chromatography that is performed on cellulose columns in the form of a paper sheet. Cellulose contains a large amount of bound water even when extensively dried. Partitioning occurs between the bound water and the developing solvent. Frequently, the solvent used is water. Usually, very small volumes of the solution mixture to be separated is placed at top of the paper and allowed to dry. Capillary action draws the solvent through the paper, dissolves the sample, and moves the components in the direction of flow. Paper chromatograms may be developed for either ascending or descending solvent flow. Two dimensional separations are permitted by changing the axis of migration 90.degree. after the first run.

Thin layer chromatography (TLC) is very commonly used to separate lipids and, therefore, is considered a preferred embodiment of the present invention. TLC has the advantages of paper chromatography, but allows the use of any substance that can be finely divided and formed into a uniform layer. In TLC, the stationary phase is a layer of sorbent spread uniformly over the surface of a glass or plastic plate. The plates are usually made by forming a slurry of sorbent that is poured onto the surface of the gel after creating a well by placing tape at a selected height along the perimeter of the plate. After the sorbent dries, the tape is removed and the plate is treated just as paper in paper chromatography. The sample is applied and the plate is contacted with a solvent. Once the solvent has almost reached the end of the plate, the plate is removed and dried. Spots can then be identified by fluorescence, immunologic identification, counting of radioactivity, or by spraying varying reagents onto the surface to produce a color change.

In Gas-Liquid chromatography (GLC), the mobile phase is a gas and the stationary phase is a liquid adsorbed either to the inner surface of a tube or column or to a solid support. The liquid usually is applied as a solid dissolved in a volatile solvent such as ether. The sample, which may be any sample that can be volatized, is introduced as a liquid with an inert gas, such as helium, argon or nitrogen, and then heated. This gaseous mixture passes through the tubing. The vaporized compounds continually redistribute themselves between the gaseous mobile phase and the liquid stationary phase, according to their partition coefficients.

The advantage of GLC is in the separation of small molecules. Sensitivity and speed are quite good, with speeds that approach 1000 times that of standard liquid chromatography. By using a non-destructive detector, GLC can be used preparatively to purify grams quantities of material. The principal use of GLC has been in the separation of alcohols, esters, fatty acids and amines.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

The gel material for gel chromatography is a three-dimensional network whose structure is usually random. The gels consist of cross-linked polymers that are generally inert, do not bind or react with the material being analyzed, and are uncharged. The space filled within the gel is filled with liquid and this liquid occupies most of the gel volume. Common gels are dextran, agarose and polyacrylamide; they are used for aqueous solution.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography.

11. Mutagenesis

Other embodiments include assays or compositions involving mutagenized PSGs or constructs expressing mutagenized PSGs. Where employed, mutagenesis will be accomplished by a variety of standard, mutagenic procedures. Mutation is the process whereby changes occur in the quantity or structure of an organism. Mutation can involve modification of the nucleotide sequence of a single gene, blocks of genes or whole chromosome. Changes in single genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

11.1. Random Mutagenesis

11.1.1 Insertional Mutagenesis

Insertional mutagenesis is based on the inactivation of a gene via insertion of a known DNA fragment. Because it involves the insertion of some type of DNA fragment, the mutations generated are generally loss-of-function, rather than gain-of-function mutations. However, there are several examples of insertions generating gain-of-function mutations. Insertion mutagenesis has been very successful in bacteria and Drosophila and recently has become a powerful tool in corn; Arabidopsis; and Antirrhinum.

Transposable genetic elements are DNA sequences that can move (transpose) from one place to another in the genome of a cell. The first transposable elements to be recognized were the Activator/Dissociation elements of Zea mays. Since then, they have been identified in a wide range of organisms, both prokaryotic and eukaryotic.

Transposable elements in the genome are characterized by being flanked by direct repeats of a short sequence of DNA that has been duplicated during transposition and is called a target site duplication. Virtually all transposable elements whatever their type, and mechanism of transposition, make such duplications at the site of their insertion. In some cases the number of bases duplicated is constant, in other cases it may vary with each transposition event. Most transposable elements have inverted repeat sequences at their termini. These terminal inverted repeats may be anything from a few bases to a few hundred bases long and in many cases they are known to be necessary for transposition.

Eukaryotic elements can be classified according to their structure and mechanism of transportation. The primary distinction is between elements that transpose via an RNA intermediate, and elements that transpose directly from DNA to DNA.

Elements that transpose via an RNA intermediate often are referred to as retrotransposons, and their most characteristic feature is that they encode polypeptides that are believed to have reverse transcriptionase activity. There are two types of retrotransposon. Some resemble the integrated proviral DNA of a retrovirus in that they have long direct repeat sequences, long terminal repeats (LTRs), at each end. The similarity between these retrotransposons and proviruses extends to their coding capacity. They contain sequences related to the gag and pol genes of a retrovirus, suggesting that they transpose by a mechanism related to a retroviral life cycle. Retrotransposons of the second type have no terminal repeats. They also code for gag- and pol-like polypeptides and transpose by reverse transcription of RNA intermediates, but do so by a mechanism that differs from that of retrovirus-like elements. Transposition by reverse transcription is a replicative process and does not require excision of an element from a donor site.

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another.

11.1.2 Chemical Mutagenesis

Chemical mutagenesis offers certain advantages, such as the ability to find a full range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the O4 position of thymine induced by exposure to n-nitrosoureas results in TA to CG transitions.

A high correlation between mutagenicity and carcinogenity is the underlying assumption behind the Ames test which speedily assays for mutants in a bacterial system, together with an added rat liver homogenate, which contains the microsomal cytochrome P450, to provide the metabolic activation of the mutagens where needed.

In vertebrates, several carcinogens have been found to produce mutation in the ras proto-oncogene. N-nitroso-N-methyl urea induces mammary, prostate and other carcinomas in rats with the majority of the tumors showing a G to A transition at the second position in codon 12 of the Ha-ras oncogene. Benzo[a]pyrene-induced skin tumors contain A to T transformation in the second codon of the Ha-ras gene.

11.1.3 Radiation Mutagenesis

The integrity of biological molecules is degraded by the ionizing radiation. Adsorption of the incident energy leads to the formation of ions and free radicals, and breakage of some covalent bonds. Susceptibility to radiation damage appears quite variable between molecules, and between different crystalline forms of the same molecule. It depends on the total accumulated dose, and also on the dose rate (as once free radicals are present, the molecular damage they cause depends on their natural diffusion rate and thus upon real time). Damage is reduced and controlled by making the sample as cold as possible.

Ionizing radiation causes DNA damage and cell killing, generally proportional to the dose rate. Ionizing radiation has been postulated to induce multiple biological effects by direct interaction with DNA, or through the formation of free radical species leading to DNA damage (Hall, 1988). These effects include gene mutations, malignant transformation, and cell killing. Although ionizing radiation has been demonstrated to induce expression of certain DNA repair genes in some prokaryotic and lower eukaryotic cells, little is known about the effects of ionizing radiation on the regulation of mammalian gene expression. Several studies have described changes in the pattern of protein synthesis observed after irradiation of mammalian cells. For example, ionizing radiation treatment of human malignant melanoma cells is associated with induction of several unidentified proteins. Synthesis of cyclin and co-regulated polypeptides is suppressed by ionizing radiation in rat REF52 cells, but not in oncogene-transformed REF52 cell lines. Other studies have demonstrated that certain growth factors or cytokines may be involved in x-ray-induced DNA damage. In this regard, platelet-derived growth factor is released from endothelial cells after irradiation.

The term “ionizing radiation” means radiation comprising particles or photons that have sufficient energy or can produce sufficient energy via nuclear interactions to produce ionization (gain or loss of electrons). An exemplary and preferred ionizing radiation is an x-radiation. The amount of ionizing radiation needed in a given cell generally depends upon the nature of that cell. Typically, an effective expression-inducing dose is less than a dose of ionizing radiation that causes cell damage or death directly. Means for determining an effective amount of radiation are well known in the art.

In certain embodiments, an effective expression inducing amount is from about 2 to about 30 Gray (Gy) administered at a rate of from about 0.5 to about 2 Gy/minute. Even more preferably, an effective expression inducing amount of ionizing radiation is from about 5 to about 15 Gy. In other embodiments, doses of 2-9 Gy are used in single doses. An effective dose of ionizing radiation may be from 10 to 100 Gy, with 15 to 75 Gy being preferred, and 20 to 50 Gy being more preferred.

Any suitable means for delivering radiation to a tissue may be employed in the present invention in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

11.1.4 In Vitro Scanning Mutagenesis

Random mutagenesis also may be introduced using error prone PCR. The rate of mutagenesis may be increased by performing PCR in multiple tubes with dilutions of templates.

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation.

In recent years, techniques for estimating the equilibrium constant for ligand binding using minuscule amounts of protein have been developed. The ability to perform functional assays with small amounts of material can be exploited to develop highly efficient, in vitro methodologies for the saturation mutagenesis of antibodies. Cloning steps can be bypassed by combining PCR mutagenesis with in vitro transcription/translation for the high throughput generation of protein mutants. Here, the PCR products are used directly as the template for the in vitro transcription/translation of the mutant protein. Because of the high efficiency with which all 19 amino acid substitutions can be generated and analyzed in this way, it is now possible to perform saturation mutagenesis on numerous residues of interest, a process that can be described as in vitro scanning saturation mutagenesis.

In vitro scanning saturation mutagenesis provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

11.1.5 Random Mutagenesis by Fragmentation and Reassembly

A method for generating libraries of displayed polypeptides is described in U.S. Pat. No. 5,380,721. The method comprises obtaining polynucleotide library members, pooling and fragmenting the polynucleotides, and reforming fragments therefrom, performing PCR amplification, thereby homologously recombining the fragments to form a shuffled pool of recombined polynucleotides.

11.2 Site-Directed Mutagenesis

Structure-guided site-specific mutagenesis represents a powerful tool for the dissection and engineering of protein-ligand interactions. The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting. Hundreds, and possibly even thousands, of site specific mutants must be studied. However, improved techniques make production and rapid screening of mutants much more straightforward.

12. Rational Drug Design

The goal of rational drug design is to produce structural analogs of biologically active compounds. By creating such analogs, it is possible to fashion drugs which are more active or stable than the natural molecules, which have different susceptibility to alteration or which may affect the function of various other molecules. In one approach, one would generate three-dimensional structures for PSG and a modulator of PSG or a fragment thereof. This could be accomplished by X-ray crystallography, computer modeling or by a combination of both approaches. An alternative approach, involves the random replacement of functional groups throughout the PSG or a modulator of PSG, and the resulting affect on function determined.

It also is possible to isolate a PSG or a modulator of PSG by antibody capture, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

13. Inhibitory Nucleic Acids Specific for PSG

The inhibitory nucleic acids of certain embodiments of the present disclosure are directed to inhibiting or interfering with the expression of PSG, an isoform, or mutation thereof, including, but not limited, PSG1-11 (SEQ ID NOs 1-11), and combinations thereof. The sequences of PSG isoforms are known in the art, and can be found for example in Genbank which sequences are incorporated by reference herein in their entirety. SEQ ID Nos: 1-11 correspond to the protein sequences of PSG1-11, respectively. The corresponding nucleic acid sequences can be extrapolated therefrom by those of ordinary skill in the art. The inhibitory nucleic acids disclosed herein include small inhibitory ribonucleic acids (siRNAs) that are typically less than 30 nucleotides in length, more typically 21 to 23 nucleotides in length, and can be single or double stranded and specifically bind to mRNA encoding PSG1-11. One strand of a double-stranded siRNA comprises at least a partial sequence complementary to a target mRNA, for example a PSG mRNA. The ribonucleotides of the siRNA can be natural or artificial and can be chemically modified. Longer siRNAs can comprise cleavage sites that can be enzymatically or chemically cleaved to produce siRNAs having lengths less than 30 nucleotides. siRNAs share sequence homology with corresponding target mRNAs. The phosphate backbones of the siRNAs can be chemically modified to resist enzymatic degradation. The sequence homology can be about 100 percent or less, but sufficient to result in sequence specific association between the siRNA and the targeted mRNA.

Nucleic acids, in particular RNA, are known to participate in a form of post-transcriptional gene silencing termed “RNA interference” or RNAi. First observed in plants, reduction of expression of specific mRNA sequences was found to be inducible in Drosophila melanogaster and Caenorhabditis elegans by introduction of double-stranded RNA (dsRNA) molecules mimicking the sequence of the mRNA. The effect was found to be potent and extremely long-lived in these experimental model organisms, generally extending to the F1 progeny of a treated adult specimen. Additionally, the effect was found to be exquisitely sequence-specific; discrepancy of even a few base pairs between the dsRNA and the target mRNA virtually abolished the silencing. RNAi has been used experimentally in these non-mammalian systems to generate transient silencing of specific genes of interest, especially those which are not amenable to more traditional gene knockout methods (e.g., those that produce embryonic lethality and thus cannot be studied in the adult animal).

The first evidence that dsRNA could lead to gene silencing came from work in the nematode Caenorhabditis elegans. Researchers Guo and Kemphues used antisense RNA to shut down expression of the par-1 gene in order to assess its function. As expected, injection of the antisense RNA disrupted expression of par-1, but quizzically, injection of the sense-strand control did too. This result was a puzzle until three years later. It was then that Fire and Mello first injected dsRNA—a mixture of both sense and antisense strands—into C. elegans. This injection resulted in much more efficient silencing than injection of either the sense or the antisense strands alone. Injection of just a few molecules of dsRNA per cell was sufficient to completely silence the homologous gene's expression. Furthermore, injection of dsRNA into the gut of the worm caused gene silencing not only throughout the worm, but also in its first generation offspring. The potency of RNAi inspired Fire and Timmons to try feeding nematodes bacteria that had been engineered to express dsRNA homologous to the C. elegans unc-22 gene. Surprisingly, these worms developed an unc-22 null-like phenotype. Further work showed that soaking worms in dsRNA was also able to induce silencing. These strategies, whereby large numbers of nematodes are exposed to dsRNA, have enabled large-scale screens to select for RNAi-defective C. elegans mutants and have led to large numbers of gene knockout studies within this organism. Thus, one embodiment of the present invention discloses siRNAs comprising a sense strand and an anti-sense strand, wherein the sense strand comprises at least a partial sequence of a target mRNA.

RNAi has also been observed in Drosophila melanogaster. Although a strategy in which yeast were engineered to produce dsRNA and then fed to fruit flies failed to work, microinjecting Drosophila embryos with dsRNA does induce silencing. Silencing can also be induced by biolistic techniques in which dsRNA is “shot” into Drosophila embryos, or by engineering flies to carry DNA containing an inverted repeat of the gene to be silenced. Over the last few years, these RNAi strategies have been used as reverse genetics tools in Drosophila organisms, embryo lysates, and cells to characterize various loss-of-function phenotypes. Zamore and colleagues found that dsRNA added to Drosophila embryo lysates was processed to 21-23 nucleotide species. They also found that the homologous endogenous mRNA was cleaved only in the region corresponding to the introduced dsRNA and that cleavage occurred at 21-23 nucleotide intervals.

Current models of RNAi divide the process of inhibition into broad “initiation” and “effector” stages. In the initiation step, input dsRNA is digested into 21-23 nucleotide small interfering RNAs (siRNAs), which have also been called “guide RNAs.” Evidence indicates that siRNAs are produced when the enzyme Dicer, a member of the RNase III family of dsRNA-specific ribonucleases, processively cleaves dsRNA in an ATP-dependent, processive manner. Successive cleavage events degrade the RNA to 19-21 bp duplexes (siRNAs), each with 2-nucleotide 3′ overhangs. Inhibitory nucleic acids of the present invention can be enzymatically cleaved, for example in vivo, to produce siRNAs from 10 to about 30 nucleotides, typically about 19 to about 23 nucleotides.

In the effector step, the siRNA duplexes bind to a nuclease complex to form what is known as the RNA-induced silencing complex, or RISC. An ATP-depending unwinding of the siRNA duplex is required for activation of the RISC. The active RISC then targets the homologous transcript by base pairing interactions and cleaves the mRNA ˜12 nucleotides from the 3′ terminus of the siRNA. Although the mechanism of cleavage is at this date unclear, research indicates that each RISC contains a single siRNA and an RNase that appears to be distinct from Dicer. Because of the remarkable potency of RNAi in some organisms, an amplification step within the RNAi pathway has also been proposed. Amplification could occur by copying of the input dsRNAs, which would generate more siRNAs, or by replication of the siRNAs themselves. Alternatively or in addition, amplification could be effected by multiple turnover events of the RISC. One embodiment encompasses the in vivo amplification of the siRNAs disclosed herein. Additionally, the siRNAs described herein can form a complex with additional proteins and/or cofactors to enzymatically cleave a target mRNA.

It will be appreciated that the siRNAs disclosed herein can be used to downregulate the expression of PSGs at the cellular level. Accordingly, PSG knockdown models systems using iRNAs can be established and utilized in the disclosed assays.

### EXAMPLES

### Exmaple 1

### Comparison of Gene Expression Between Vein and Artery to Identify Genes Contributing to Different Susceptibilities to Atherosclerosis

Vein and artery respond differently to atherogenic lesion under different conditions. Unlike arteries, veins do not develop atherosclerosis in normal anatomical locations. However, venous bypass grafts can develop accelerated atherosclerosis in vein graft disease for patients after bypass surgery. These facts suggest that in normal situations venous vascular wall could resist injuries induced by systemic atherosclerogenic factors, but in some abnormal situations (like after arterialization in vein graft disease), venous walls may be vulnerable to atherosclerosis. This could possibly be explained by the different roles of endothelial and smooth muscle cells in the development of atherosclerosis. Endothelial cells (EC) are critical in the initiation of atherosclerosis, while smooth muscle cells (SMC) are closely related to the evolution of the atheroma. The global gene expression responses to various atherogenic stimulations between human venous and arterial cells have been determined to identify genes that may contribute to different susceptibilities to atherosclerosis.

### Example 2

### High Levels of PSG Expression in Venous Endothelial and Smooth Muscle Cells in Basal Condition

When gene expression of untreated saphenous vein endothelial cells (SVEC) and coronary artery endothelial cells (CAEC) were compared by microarray analysis, five isoforms (PSG1, PSG3, PSG6, PSG7 and PSG9) out of 9 isoforms in the arrays were higher (>1.5 fold and P<0.005) in SVEC than CAEC. In untreated saphenous vein smooth muscle cells (SVSM) and coronary artery smooth muscle cells (CASM), all 9 isoforms of PSGs in the arrays are expressed higher in SVSM than CASM (See Table 1). It is unlikely a false positive result from arrays, since the result was obtained from 4 separate primary cultures from two-donors and more than 5 microarray data sets, and multiple PSG isoforms had similar changes. Therefore, for the first time, the data clearly demonstrate that PSGs are expressed in both vascular endothelial cells and smooth muscle cells. In basal state, PSG expression is higher in vein than artery.

### Example 3

### PSGs are Regulated by Atherogenic Factors in Smooth Muscle Cells

Further study shows PSG expression is modulated by different atherogenic factors in vascular cells. Oxidized LDL (OxLDL), a key atherogenic factor, pro-inflammatory cytokines TNFα and IL1β, as well as cytokines regulating cell proliferation, differentiation and migration like PDGF and TGFβ all up-regulate PSG expression in coronary arterial smooth muscle cells. No significant change of PSG expression is found in endothelial cells from both vein and artery after OxLDL, TNFα and IL1β. The data show that PSGs are not only present in SMCs, but also respond to the atherogenic stimuli in smooth muscle cells.

### Example 4

### Differential Responses of PSG Isoforms to Different Stimuli in Vascular Smooth Muscle Cells

Nine out of 11 PSG genes are present in the array. PSG 2 and PSG6 were up-regulated by all treatments (OxLDL, TNFα, IL1β, TGFβ and PDGF) in SMCs from coronary artery. However, PSG1 has opposite responses to IL1β and TGFβ (Table 1). Differential expression of PSG isoformrns in response to atherogenic stimuli suggests the different functions among the PSG isoforms.

### Example 5

### Dramatically Different Responses to Atherogenic Stimuli in Smooth Muscle Cells Between Vein and Artery

Most PSG isoforms are up-regulated in response to atherogenic stimuli in SMC from coronary artery (Table 1). Surprisingly, in SMCs from saphenous vein, most PSG isoforms are either not changed or down-regulated by the same atherogenic stimuli as arterial SMCs (Table 1). The gene expression of PSG isoforms is quite different between the two SMCs. In SMCs from saphenous vein, TGFβ and TNFα down-regulate the expression of PSG1. TGFβ also down-regulates PSG9. There was no significant change for other isoforms. The dramatic difference in PSG expressions in SMCs between vein and artery suggests that PSGs play different roles in different vascular beds.

### REFERENCES

Each of the references cited throughout the disclosure are incorporated by reference in their entirety.

1. Wurz H, Geiger W, Kunzig H J, Jabs-Lehrnann A, Bohn H, Luben G. Radioimmunoassay of SP1 (pregnancy-specific β1-glycoprotein) in maternal blood and in amniotic fluid normal and pathologic pregnancies. J Perinat Med 1981; 9 (2):67-78.

2. Tatarinov Y S, Falaleeva D M, Kalashnikov U V, Human pregnancy-specific β1-globulin and its relation to chorioepithelioma. Int J Cancer 1976; 17 (5):626-32.

3. Leslie K K, Watanabe S, Lei K J, Chou D Y, Plouzek C A, Deng H C, Torres J, Chou J Y. Linkage of two human pregnancy-specific β 1-glycoprotein genes: one is associated with hydatidiform rnole. Proc Natl Acad Sci USA 1990; 87 (15):5822-6.

4. Blomberg L A, Cohn M L, Cahill R A, Chan W Y. Effect of human pregnancy-specific β1-glycoprotein on blood cell regeneration after bone marrow transplantation, Proc Soc Exp Biol Med 1998; 217 (2):212-8.

5. Motran C C, Diaz F L, Gruppi A, Slavin D, Chatton B, Bocco X L. Human pregnancy-specific glycoprotein Ia (PSGIa) induces alternative activation in human and mouse monocytes and suppresses the accessory cell-dependent T cell proliferation, J Leukoc Biol 2002; 72(3):512-21.

6. Snyder S K, Wessner D H, Wessells J L, Waterhouse R M, Wahl L M, Zimmermann W, Dveksler G S. Pregnancy-specific glycoproteins function as immunomodulators by inducing secretion of IL-10, IL-6 and TGF-β1 by human monocytes. Am J Reprod Immunol 2001; 45 (4):205-16.

7. Bayes-Genis A, Conover C A, Overgaard M T, Bailey K R, Christiansen M, Holmes D R, Jr., Virmani R, Oxvig C, Schwartz R S, Pregnancy-associated plasma protein A as a marker of acute coronary syndromes. N Engl J Med 2001; 345 (14):1022-9.

8. Futterman L G, Lemberg L. Novel markers in the acute coronary syndrome: BNP, IL-6, PSG. Am J Crit Care 2002; 11 (2): 168-72.

9. Beaudeux J L, Burc L, Irnbert-Bismut F, Giral P, Bernard M, Bruckert E, Chapman M J. Serum plasma pregnancy-associated protein A: a potential marker of echogenic carotid atherosclerotic plaques in asymptomatic hyperlipidemic subjects at high cardiovascular risk. Arterioscier Thromb Vasc Biol 2003; 23 (1):e7-10.

10. Stulc T, Malbohan I, Malik 0.1, Fialova L, Soukupova J, Ceska R, Increased levels of pregnancy-associated plasma protein-A in patients with hypercholesterolemia: the effect of atorvastatin treatment—Am Heart J 2003; 146 (6):E21.

TABLE 1
PSG expression in basal state of endothelial and smooth muscle cells from saphenveous vein and coronary artery and in responses to different stimuli
related to atherosclerosis.

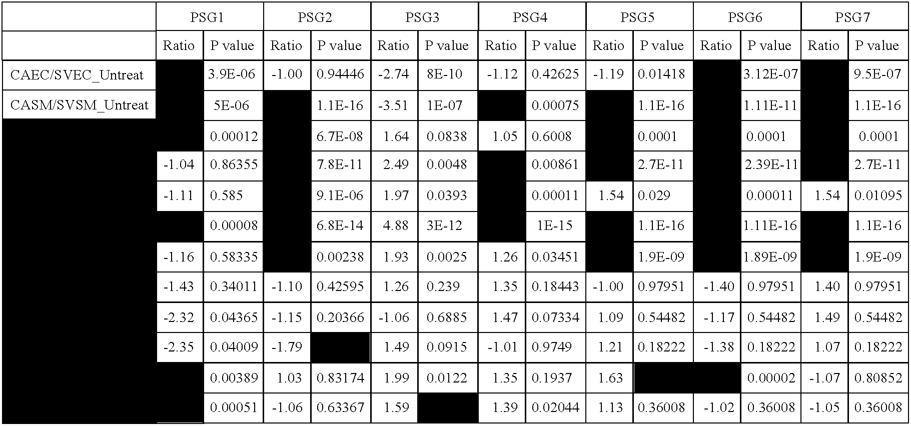

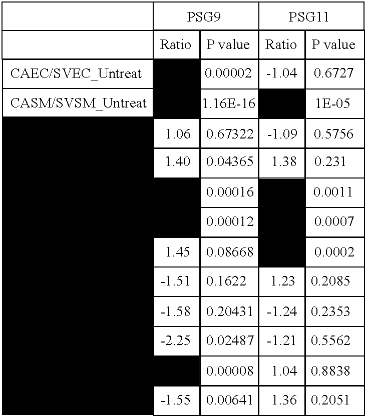

Ratio is weighted mean of 4-6 array results;
P value is derived from error-weighted one-way ANOVA. Negative fold change represents down-regulation of gene after treatment or higher gene expression level in venous vascular cells than arterial cells. Significantly changed ratios (P < 0.005 and > 1.5 fold) are shaded.

What is claimed is:

1. An array for diagnosing an inflammatory pathology, the array comprising at least one binding agent specific for a PSG polypeptide or PSG nucleic acid, isoforms, or variants thereof, wherein the at least one binding agent is bound to a surface of the array, and wherein binding of a PSG polypeptide or PSG nucleic acid, isoforms, or variants thereof to the array to the at least one binding agent in an amount less than a predetermined level is indicative of a vascular pathology.

2. The array of claim 1, wherein the binding agent is a polypeptide or polynucleotide.

3. The array of claim 2, wherein the polypeptide is a monoclonal antibody, polyclonal antibody, humanized anitbody, single chain antibody, chimeric antibody, fragment thereof, or a combination thereof.

4. The array of claim 1, further comprising a second binding agent that specifically binds a second biological marker of an inflammatory pathology.

5. The array of claim 4, wherein the second biological marker is selected from the group consisting of C-reactive protein, PAPP-A, fibrinogen, lipoprotein, interleukin-1, IL-6, neopterin, or combinations thereof.

6. A method for diagnosing an inflammatory condition, the method comprising:
a) determining the level of pregnancy-specific glycoprotein (PSG) in a biological sample from a non-pregnant patient;
b) comparing the level of PSG from the non-pregnant patient with a predetermined value of PSG indicative of healthy vasculature; and
c) diagnosing the inflammatory condition based on the level of PSG from the non-pregnant patient relative to the predetermined value of PSG indicative of healthy vasculature, wherein the patient is diagnosed as having an inflammatory condition if the level of PSG is decreased relative to that of the predetermined level of PSG indicative of healthy vasculature.

7. The method of claim 6, wherein the inflammatory condition is selected from the group consisting of atherosclerosis, rheumatoid arthritis, unstable angina, sudden cardiac death, acute myocardial infarction, Crohn's disease, vasculitis, Takayasu's arteritis, giant cell arterities, Kawasaki disease, and inflammatory bowel disease.

8. The method of claim 6, wherein the level of PSG is measured using an immunoassay.

9. The method of claim 8, wherein the immunoassay is an ELISA.

10. The method of claim 8, wherein PSG is captured with anti-PSG antibodies.

11. The method of claim 10, wherein the anti-PSG antibodies are monoclonal antibody, polyclonal antibody, humanized antibody, single chain antibody, chimeric antibody, fragments thereof, or combinations thereof.

12. The method of claim 6, wherein the biological sample is selected from the group consisting of whole blood, plasma, and serum.

13. The method of claim 6, wherein the method further comprises measuring the level of a second biological marker indicative of an inflammatory condition, and wherein the diagnosing step is based on the level of the second biological marker and the level of PSG relative to that of the predetermined value of PSG.

14. The method of claim 13, wherein the second biological marker is selected from the group consisting of high sensitivity C-reactive protein, homocysteine, fibrinogen, lipoprotein, creatine kinase MB, troponin I, troponin T, creatine kinase, creatinine, fibrinogen, interleukin-1, interleukin-6, PAPP-A, a fragment or isoform thereof, and combinations thereof.

15. A method for treating an inflammatory condition comprising administering to a mammal in need thereof an amount of a PSG modulator effective to modulate PSG expression.

16. The method of claim 15, wherein the PSG modulator modulates expression of PSG in vascular cells.

17. The method of claim 16, wherein the PSG modulator modulates expression of PSG in vascular endothelial cells or vascular smooth muscle cells.

18. The method of claim 15, wherein the PSG modulator increases expression of PSG.

19. The method of claim 18, wherein the increase in PSG expression occurs in vascular tissue.

20. The method of claim 18, wherein the modulator comprises a growth factor.

21. The method of claim 18, wherein the modulator is selected from the group consisting of TNFα, TGFβ, PDGF, IL1β, a fragment thereof, and combinations thereof.

22. A method for treating or preventing atherosclerosis comprising administering to a mammal in need thereof, a pharmaceutical composition effective to increase the expression of PSG in vascular tissue.

23. A method for monitoring the effectiveness of a therapy for an inflammatory pathology, the method comprising:
(a) administering a therapeutic agent to a host over a period of time; and
(b) determining expression levels of PSG in the host's vascular tissue after administration of the therapeutic agent, wherein an increase in the expression levels of PSG in the host's vascular tissue after administering the therapeutic agent indicates the therapeutic agent is effective.

24. A method for determining a predisposition for vascular pathology, the method comprising:
comparing levels of PSG expression in vascular tissue of a host with a predetermined value indicative of healthy vascular tissue, wherein levels of PSG expression in the host less than the predetermined value is indicative of a disposition for developing a vascular pathology.


SEQUENCE LISTING (11 sequences)

SEQ ID NO: 1 - protein, 426 aa, Homo sapiens
MGTLSAPPCTQRIKWKGLLLTASLLNFWNLPTTAQVTIEAEPTKVSEGKDVLLLVHNLPQNLTGYIWYKGQMRDLYHYITSYVVDGEIIIYGPAYSGRETAYSNASLLIQNVTREDAGSYTLHIIKGDDGTRGVTGRFTFTLHLETPKPSISSSNLNPRETMEAVSLTCDPETPDASYLWWMNGQSLPMTHSLKLSETNRTLFLLGVTKYTAGPYECEIRNPVSASRSDPVTLNLLPKLPKPYITINNLNPRENKDVLNFTCEPKSENYTYIWWLNGQSLPVSPRVKRPIENRILILPSVTRNETGPYQCEIRDRYGGIRSDPVTLNVLYGPDLPRIYPSFTYYRSGEVLYLSCSADSNPPAQYSWTINEKFQLPGQKLFIRHITTKHSGLYVCSVRNSATGKESSKSMTVEVSGKWIPASLAIGF

SEQ ID NO: 2 - protein, 335 aa, Homo sapiens
MGPLSAPPCTEHIKWKGLLLTASLLNFWNLPTTAQVTIEAQPPKVSEGKDVLLLVHNLPQNLTGYIWYKGQIRDLYHYITSYVVDGQIIIYGPAYSGRETAYSNASLLIQNVTREDAGSYTLHIIKRGDGTRGVTGYFTFTLYLETPKPSISSSNLNPREAMETVILTCDPETPDTSYQWWMNGQSLPMTHRFQLSETNRTLFLFGVTKYTAGPYECEIRNSGSASRSDPVTLNLLHGPDLPRIHPSYTNYRSGDNLYLSCFANSNPPAQYSWTINGKFQQSGQNLFIPQITTKHSGLYVCSVRNSATGEESSTSLTVKVSASTRIGLLPLLNPT

SEQ ID NO: 3 - protein, 428 aa, Homo sapiens
MGPLSAPPCTQRITWKGLLLTALLLNFWNLPTTAQVTIEAEPTKVSKGKDVLLLVHNLPQNLAGYIWYKGQMKDLYHYITSYVVDGQIIIYGPAYSGRETVYSNASLLIQNVTREDAGSYTLHIVKRGDGTRGETGHFTFTLYLETPKPSISSSNLYPREDMEAVSLTCDPETPDASYLWWMNGQSLPMTHSLQLSKNKRTLFLFGVTKYTAGPYECEIRNPVSASRSDPVTLNLLPKLPKPYITINNLNPRENKDVLAFTCEPKSENYTYIWWLNGQSLPVSPRVKRPIENRILILPSVTRNETGPYQCEIQDRYGGIRSYPVTLNVLYGPDLPRIYPSFTYYHSGENLYLSCFADSNPPAEYSWTINGKFQLSGQKLFIPQITTKHSGLYACSVRNSATGMESSKSMTVEVSAPSGTGHLPGLNPL

SEQ ID NO: 4 - protein, 419 aa, Homo sapiens
MGPLSAPPCTHLITWKGVLLTASLLNFWNPPTTAQVTIEAQPPKVSEGKDVLLLVHNLPQNLAGYIWYKGQMTYLYHYITSYVVDGQRIIYGPAYSGRERVYSNASLLIQNVTQEDAGSYTLHIIKRRDGTGGVTGHFTFTLHLETPKPSISSSNLNPREAMEAVILTCDPATPAASYQWWMNGQSLPMTHRLQLSKTNRTLFIFGVTKYIAGPYECEIRNPVSASRSDPVTLNLLPKLPKPYITINNLNPRENKDVLTFTCEPKSENYTYIWWLNGQSLPVSPRVKRPIENRILILPNVTRNETGPYQCEIRDRYGGIRSDPVTLNVLYGPDLPSIYPSFTYYRSGENLYLSCFAESNPRAQYSWTINGKFQLSGQKLSIPQITTKHSGLYACSVRNSATGKESSKSITVKVSDWILP

SEQ ID NO: 5 - protein, 335 aa, Homo sapiens
MGPLSAPPCTQHITWKGVLLTASLLNFWNLPITAQVTIEALPPKVSEGKDVLLLVHNLPQNLAGYIWYKGQLMDLYHYITSYVVDGQINIYGPAYTGRETVYSNASLLIQNVTREDAGSYTLHIIKRGDRTRGVTGYFTFNLYLKLPKPYITINNSKPRENKDVLAFTCEPKSENYTYIWWLNGQSLPVSPRVKQPIENRILILPSVTRNETGPYECEIRDRDGGMHSDPVTLNVLYGPDLPSIYPSFTYYRSGENLYLSCFAESNPPAEYFWTINGKFQQSGQKLSIPQITTKHRGLYTCSVRNSATGKESSKSMTVEVSAPSGIGRLPLLNPI

SEQ ID NO: 6 - protein, 424 aa, Homo sapiens
MGPLSAPPCTQHITWKGLLLTASLLNFWNLPTTAQVIIEAKPPKVSEGKDVLLLVHNLPQNLTGYIWYKGQMTDLYHYITSYVVHGQIIYGPAYSGRETVYSNASLLIQNVTQEDAGSYTLHIIKRGDGTGGVTGYFTVTLYSETPKPSISSSNLNPREVMEAVRLICDPETPDASYLWLLNGQNLPMTHRLQLSKTNRTLYLFGVTKYIAGPYECEIRNPVSASRSDPVTLNLLPKLPMPYITINNLNPREKKDVLAFTCEPKSRNYTYIWWLNGQSLPVSPRVKRPIENRILILPSVTRNETGPYQCEIRDRYGGIRSNPVTLNVLYGPDLPRIYPSFTYYRSGENLDLSCFADSNPPAEYSWTINGKFQLSGQKLFIPQITTNHSGLYACSVRNSATGKEISKSMIVKVSGPCHGNQTESH

SEQ ID NO: 7 - protein, 419 aa, Homo sapiens
MGPLSAPPCTQHITWKGLLLTASLLNFWNPPTTAQVTIEAQPPKVSEGKDVLLLVHNLPQNLTGYIWYKGQIRDLYHYVTSYVVDGQIIKYGPAYSGRETVYSNASLLIQNVTQEDTGSYTLHIIKRGDGTGGVTGRFTFTLYLETPKPSISSSNFNPREATEAVILTCDPETPDASYLWWMNGQSLPMTHSLQLSETNRTLYLFGVTNYTAGPYECEIRNPVSASRSDPVTLNLLPKLPKPYITINNLNPRENKDVSTFTCEPKSENYTYIWWLNGQSLPVSPRVKRRIENRILILPSVTRNETGPYQCEIRDRYGGIRSDPVTLNVLYGPDLPRIYPSFTYYHSGQNLYLSCFADSNPPAQYSWTINGKFQLSGQKLSIPQITTKHSGLYACSVRNSATGKESSKSVTVRVSDWTLP

SEQ ID NO: 8 - protein, 144 aa, Homo sapiens
MGLLSAPPCTQRITWKGLLLTASLLNFWNPPTTAQVTIEAQPTKVSEGKDVLLLVHNLPQNLTGYIWYKGQIRDLYHYITSYVVDGQIIIYGPAYSGRETIYSNASLLIQNVTQEDAGSYTLHIIMGGDENRGVTGHFTFTLYR

SEQ ID NO: 9 - protein, 426 aa, Homo sapiens
MGPLPAPSCTQRITWKGLLLTASLLNFWNPPTTAEVTIEAQPPKVSEGKDVLLLVHNLPQNLPGYFWYKGEMTDLYHYIISYIVDGKIIIYGPAYSGRETVYSNASLLIQNVTRKDAGTYTLHIIKRGDETREEIRHFTFTLYLETPKPYISSSNLNPREAMEAVRLICDPETLDASYLWWMNGQSLPVTHRLQLSKTNRTLYLFGVTKYIAGPYECEIRNPVSASRSDPVTLNLLPKLPIPYITINNLNPRENKDVLAFTCEPKSENYTYIWWLNGQSLPVSPGVKRPIENRILILPSVTRNETGPYQCEIQDRYGGLRSNPVILNVLYGPDLPRIYPSFTYYRSGENLDLSCFTESNPPAEYFWTINGKFQQSGQKLFIPQITRNHSGLYACSVHNSATGKEISKSMTVKVSGPCHGDLTESQS

SEQ ID NO: 10 - protein, 424 aa, Homo sapiens [sequence omitted]

SEQ ID NO: 11 - protein, 213 aa, Homo sapiens [sequence omitted]